(19) United States
(12) Patent Application Publication
(10) Pub. No.: US 2017/0204024 A1
(43) Pub. Date: Jul. 20, 2017

(54) Process for the production of high-purity paraxylene based on a xylene cut, a process using one simulated mobile bed separation unit and two isomerization units, one in gas phase and the other in liquid phase

(71) Applicant: IFP Energies Nouvelles, Rueil-Malmaison Cedex (FR)

(72) Inventors: Heloise DREUX, Lyon (FR); Philibert LEFLAIVE, Mions (FR); Damien LEINEKUGEL LE COCQ, Oullins (FR)

(73) Assignee: IFP Energies Nouvelles, Rueil-Malmaison Cedex (FR)

(21) Appl. No.: 15/326,699

(22) PCT Filed: Jun. 10, 2015
(86) PCT No.: PCT/EP2015/062985

§ 371 Date: Jan. 17, 2017

(30) Foreign Application Priority Data
Jul. 18, 2014 (FR) 1456942

(51) Int. Cl. C07C 5/27

(52) U.S. Cl. CPC C07C 5/2737; C07C 5/2775; C07C 2529/40; C07C 2529/74

(57) ABSTRACT

The present invention describes a process for the production of high-purity paraxylene based on a xylene cut, a process using one simulated mobile bed separation unit and two isomerization units, one in gas phase and the other in liquid phase.

### FIELD OF THE INVENTION

Paraxylene production has increased constantly for thirty years. Paraxylene is used mainly for the production of terephthalic acid and polyethylene terephthalate resins, in order to provide synthetic textiles, bottles, and plastic materials more generally.

In order to satisfy the ever-increasing demand for paraxylene, petrochemists have a choice between increasing the capacity of existing units or constructing new units.

The present invention describes a process for the production of high-purity paraxylene which can be applied equally well to new units and to the debottlenecking of existing units.

### EXAMINATION OF THE PRIOR ART

The production of high-purity paraxylene by separation by adsorption is well known from the prior art. Industrially, this operation is carried out within a sequence of processes called “C8 aromatic loop”. This “C8 aromatic loop” includes a stage of elimination of the heavy compounds (i.e. C9+) in a distillation column called “xylenes column”. The top flow of this column, which contains the C8-aromatic isomers, is then sent to the process for the separation of the paraxylene, which is very generally a process of separation by adsorption in a simulated moving bed.

The extract which contains the paraxylene is then distilled (extraction column, then toluene column), in order to obtain high-purity paraxylene. The raffinate, rich in metaxylene, orthoxylene and ethylbenzene, after a stage of elimination of the solvent by distillation, is treated in a catalytic isomerization unit which returns a mixture of C8 aromatics, in which the proportion of xylenes (ortho-, meta-, para-xylenes) is practically at thermodynamic equilibrium, and the quantity of ethylbenzene reduced. This mixture is again sent to the “xylenes column” with the fresh feedstock.

All the industrial processes for the isomerization of the C8-aromatics make it possible to isomerize the xylenes. On the other hand, the conversion of the ethylbenzene depends on the type of process and catalyst selected. In fact the petrochemical complexes use a so-called “isomerizing” (i.e. isomerizing ethylbenzene to a mixture of C8-aromatics) or “dealkylating” (dealkylation of ethylbenzene to benzene) isomerization unit, in order to favour the production either of paraxylene alone, or of benzene and paraxylene respectively.

The choice of catalyst used depends on the desired conversion of the ethylbenzene. When the target reaction is the isomerization of the ethylbenzene, it requires a bi-functional catalyst having both an acid function and a hydrogenating function.

It has in fact been demonstrated that the ethylbenzene is first hydrogenated to ethylcyclohexane on the metallic sites, then converted to dimethylcyclohexene on acid sites by contraction then expansion of the ring, and finally dehydrogenated to xylenes.

When the target reaction is the dealkylation of the ethylbenzene, it is produced only on the acid sites. However, the presence of a hydrogenating phase on the catalyst makes it possible to immediately hydrogenate the ethylene formed and to obtain complete dealkylation, thus avoiding any subsequent realkylation. In both cases, the incorporation of a metallic phase in the catalyst also makes it possible to ensure the stability thereof.

The industrial isomerization processes therefore use bifunctional heterogeneous catalysts (acid and metallic) utilized in a fixed bed and operating in vapour phase under hydrogen pressure, in temperature ranges generally comprised between 380-440° C. and pressures from 10 to 20 bar.

The choice of an “isomerizing” isomerization makes it possible, as indicated above, to maximize the production of paraxylene, which is the compound having the highest added value at the aromatic complex outlet. This solution however has the drawback of generating, during the isomerization stage, losses of aromatic rings by cracking that are greater than with a dealkylating isomerization, the ring being temporarily at least partially hydrogenated.

The choice of the type of isomerization is therefore presented as a compromise between, on the one hand, the minimization of the loss of aromatic rings associated with a coproduction of benzene, a product with a lower added value than paraxylene (dealkylating isomerization) and, on the other hand, a maximization of the paraxylene production which has the drawback of generating greater losses of aromatic rings (“isomerizing” isomerization).

There is therefore a need for a process layout allowing both a maximization of the quantity of paraxylene produced and a reduced loss of aromatic rings.

Several solutions are proposed in the prior art for achieving this objective; these generally implement an isomerization (preferably dealkylating), combined with stages for the conversion of benzene by transalkylation and/or methylation of toluene or of benzene, such as for example in US2013/0267746.

It has surprisingly been discovered that the combination, within an aromatic complex, of an “isomerizing” isomerization and a liquid-phase isomerization as described for example in patents US2011/263918, U.S. Pat. No. 7,371,913, U.S. Pat. No. 4,962,258 and U.S. Pat. No. 6,180,550 made it possible to maximize the quantity of paraxylene produced while having a reduced loss of aromatic rings with respect to an aromatic complex according to the prior art.

Document US 2014/0155667 describes a process for the production of paraxylene comprising a xylenes separation unit and two isomerization units combined so as to reduce the recycling of xylenes.

No operating condition is provided for the isomerization units. Document FR 2 862 638 describes a process for the production of paraxylene also using a xylenes separation unit and two isomerization units, the separation unit producing two raffinates. In this document, the operating conditions of the two isomeration units are not differentiated.

### BRIEF DESCRIPTION OF THE FIGURES

FIG. 1 shows a layout of the process according to the present invention.

FIG. 2 shows a layout of the process according to the prior art.

In the remainder of the text the simulated moving bed separation unit (abbreviation SMB) is referred to as separation unit (SMB), and the two isomerization units as (ISOM-1) and (ISOM-2). The columns (S-1), (RAF) and (EXT) are distillation columns.

### BRIEF DESCRIPTION OF THE INVENTION

The present invention can be defined as a process for the production of high-purity paraxylene based on a xylenes cut containing ethylbenzene and C9+ compounds, a process using one simulated moving bed separation unit (SMB) and two isomerization units, one (ISOM-1) operating in liquid phase, and the other (ISOM-2) operating in gas phase.

The process according to the invention consists of the following series of stages:
the fresh feedstock (1) in a mixture with the isomerate (16) originating from the gas-phase isomerization unit (ISOM-2) is sent into the distillation column (S-1) from which a flow (3) which is mixed with the second isomerate (14) originating from the liquid-phase isomerization unit (ISOM-1) is removed at the top, and a flow (4) essentially constituted by C9 and C10 aromatic compounds and optionally orthoxylene is removed at the bottom.
a simulated mobile bed separation of the flow (5) resulting from the mixture of the flows (3) and (14) is carried out in a separation unit (SMB) comprising at least one adsorber containing a plurality of interconnected beds and operating in a closed loop, said separation unit comprising at least four zones defined as follows:
zone 1 comprised between the injection of the desorbent (11) and the draw-off of the extract (6),
zone 2 comprised between the draw-off of the extract (6) and the injection of the feedstock (5),
zone 3 comprised between the injection of the feedstock (5) and the draw-off of the raffinate (9),
zone 4 comprised between the draw-off of the raffinate (9) and the injection of the desorbent (11),
the extract 6 is sent into a distillation column (EXT), from which a mixture of paraxylene and toluene is drawn off at the top through the line (7) and the desorbent (8) which is sent back into the separation unit (SMB) is drawn off at the bottom through the line (11).
the raffinate 9 is sent into a distillation column (RAF), from which the desorbent (10) which is sent back into the separation unit (SMB) through the line (11) is drawn off at the bottom, and a mixture of metaxylene, orthoxylene and ethylbenzene which is sent through a line (12) to the isomerization units (ISOM-1 and ISOM-2) is drawn off at the top,
a first part of the flow (12), denoted flow (13), is sent into the liquid-phase isomerization unit (ISOM-1), in order to obtain a first isomerate (14), partially supplying the simulated moving bed separation unit (SMB),
a second part of the flow (12), denoted flow (15), is sent into the gas-phase isomerization unit (ISOM-2), in order to obtain an isomerate (16), which is sent in a mixture with the fresh feedstock (1) into the distillation column (S-1).

The gas-phase isomerization unit (ISOM-2) operates under the following conditions:
temperature greater than 300° C., preferably from 350° C. to 480° C.,
pressure less than 4.0 MPa and preferably from 0.5 to 2.0 MPa,
hourly space velocity less than 10 h⁻¹, preferably comprised between 0.5 h⁻¹ and 6 h⁻¹,
hydrogen to hydrocarbon molar ratio less than 10, and preferably comprised between 3 and 6.

and the catalyst used in said isomerization unit ISOM-2 comprising at least one zeolite having channels the opening of which is defined by a ring with 10 to 12 oxygen atoms (10 MR or 12 MR), and at least one group VIII metal at a content comprised between 0.1 and 0.3% by weight, inclusive.

According to a preferred variant of the process for the production of high-purity paraxylene according to the present invention, the isomerization unit (ISOM-1) operates in liquid phase under the following conditions:
Temperature less than 300° C., preferably 200° C. to 260° C.,
Pressure less than 4 MPa, preferably 2 to 3 MPa,
Hourly space velocity (HSV) less than 10 h⁻¹ (10 litres per litre per hour), preferably comprised between 2 and 4 h⁻¹,
Catalyst comprising at least one zeolite having channels the opening of which is defined by a ring with 10 or 12 oxygen atoms (10 MR or 12 MR), preferentially a catalyst comprising at least one zeolite having channels the opening of which is defined by a ring with 10 oxygen atoms (10 MR), and even more preferably, a catalyst comprising a zeolite of ZSM-5 type.

According to another variant of the process for the production of high-purity paraxylene according to the invention, the catalyst used in the isomerization unit (ISOM-2) contains from 1 to 70% by weight of a zeolite of the EUO structure type (EU-1 for example) comprising silicon and at least one element T preferably selected from aluminium and boron, the Si/T ratio of which is comprised between 5 and 100.

According to another preferred variant of the process for the production of high-purity paraxylene according to the invention, the zeolite forming part of the catalyst of the isomerization unit (ISOM-2) is at least partially in the form of hydrogen, and the sodium content is such that the Na/T atomic ratio is less than 0.1.

According to another preferred variant of the process for the production of high-purity paraxylene according to the invention, the catalyst of the isomerization unit (ISOM-2) can contain between 0.01 and 2% by weight of tin or indium, and sulphur at a content of 0.5 to 2 atoms per atom of the group VIII metal.

According to another preferred variant of the process for the production of high-purity paraxylene according to the invention, the total number of beds of the separation unit (SMB) is comprised between 6 and 24 beds, and preferably between 8 and 15 beds, distributed over one or more adsorbers, the number of beds being adjusted so that each bed has a height comprised between 0.70 m and 1.40 m.

According to another preferred variant of the process for the production of high-purity paraxylene according to the invention, the distribution of the quantity of solid adsorbent in each zone of the separation unit (SMB) is as follows:
the quantity of solid adsorbent in zone 1 is 17%±5%,
the quantity of solid adsorbent in zone 2 is 42%±5%,
the quantity of solid adsorbent in zone 3 is 25%±5%,
the quantity of solid adsorbent in zone 4 is 17%±5%,

According to another preferred variant of the process for the production of high-purity paraxylene according to the invention, the desorbent and the feedstock are injected into the separation unit (SMB, with a ratio by volume of at most 1.7/1 and preferably comprised between 1.5/1 and 0.4/1, inclusive.

According to another preferred variant of the process for the production of high-purity paraxylene according to the invention, not only one single raffinate (9) but two distinct raffinates (R1) and (R2), i.e. collected from two different points of the unit (SMB), are extracted from the separation unit (SMB), (R1) being sent to the isomerization unit (ISOM-1) and (R2) being sent into the isomerization unit (ISOM-2).

### DETAILED DESCRIPTION OF THE INVENTION

The feedstock (1) is mixed with the isomerate (16) in order to form the flow (2). The flow (2) is sent into a distillation column (S-1) from where a mixture (3) the major part comprising metaxylene, paraxylene, ethylbenzene, and at least a part of orthoxylene is drawn off at the top, and from where a flow (4) of C9-C10 hydrocarbons and the remaining part of the orthoxylene is drawn off at the bottom.

The flow (3) from the top of the distillation column (S-1) is mixed with the isomerate (14) in order to form the flow (5).

A first separation of the mixture (5) is carried out in a simulated moving bed separation unit (SMB) comprising at least one adsorber containing a plurality of interconnected beds and operating in a closed loop, said separation unit comprising at least four zones delimited by the injections of the flow (5) and the desorbent (11), and the draw-offs of an extract (6) containing paraxylene, and of a raffinate (9) containing orthoxylene and metaxylene.

Preferentially, the extract (6) is distilled in a distillation column (EXT), in order to recover a first fraction (7) enriched with paraxylene,

Preferentially, the raffinate (9) is distilled in a distillation column (RAF), in order to eliminate substantially all the desorbent and in order to draw off a distilled fraction (12).

This distilled fraction (12) is divided into two flows (13) and (15). The flow (13) supplies a first isomerization unit (ISOM-1) in order to obtain a first isomerate (14) preferentially supplying the separation unit (SMB), but capable of being partially recycled to the inlet of the distillation column (S-1).

The flow (15) supplies a second isomerization unit (ISOM-2), in order to obtain a second isomerate (16), recycled to the inlet of the separation column (S-1).

The desorbent used in the separation unit (SMB) is generally selected from paradiethylbenzene, toluene, paradifluorobenzene or diethylbenzenes in a mixture. The ratio by volume of the desorbent to the feedstock in the separation unit (SMB) is comprised between 0.5 and 2.5, and preferably comprised between 0.8 and 2.

The simulated moving bed separation unit (SMB) is operated at a temperature comprised between 20° C. and 250° C., preferably between 90° C. and 210° C., and even more preferably between 140° C. and 180° C., and under a pressure comprised between the bubble pressure of xylenes at the operating temperature and 2 MPa.

The fresh feedstock is introduced through the line (1) into a distillation column (S-1). This fresh feedstock contains mainly C8-aromatic compounds, xylenes and ethylbenzene, in a variable proportion according to the origin of the cut. It can possibly contain impurities in a variable quantity depending on the origin of the feedstock which will be essentially C9 and C10 aromatic compounds and paraffinic and naphthenic compounds.

The content of naphthenic or paraffinic compounds in the feedstock is advantageously less than 1% by weight. Preferably, this content is less than 0.3% by weight, and even more preferably this content is less than 0.1% by weight.

The feedstock can originate either from a reforming unit, or from a toluene disproportionation unit, or from a unit for the transalkylation of toluene and C9 aromatics.

An isomerate conveyed by a line (16) is added to the fresh feedstock.

The bottom effluent (4) from the column (S-1) is essentially constituted by C9 and C10 aromatic compounds, and optionally orthoxylene.

Optionally, the mixture (4) of orthoxylene and C9-C10 aromatic hydrocarbons drawn off at the bottom of the distillation column (S-1), can be sent into another distillation column from which a high-purity orthoxylene flow (at least 98.5%) is extracted at the top, and a flow containing C9-C10 hydrocarbons is extracted at the bottom.

The top effluent (3) from the distillation column (S-1) is mixed with the isomerate (14) in order to form the flow (5) which constitutes the feedstock of a separation unit (SMB). The separation unit (SMB) is supplied on the one hand with the feedstock conveyed by the line (5), and on the other hand with the desorbent conveyed by a line (11).

The effluents from the separation unit (SMB) are an extract (6) and a raffinate (9), said separation unit comprising at least four zones delimited by the injections of feedstock and of desorbent, and the draw-offs of raffinate and of extract.
zone 1 comprised between the injection of the desorbent (11) and the draw-off of the extract (6),
zone 2 comprised between the draw-off of the extract (6) and the injection of the feedstock (5),
zone 3 comprised between the injection of the feedstock (5) and the draw-off of the raffinate (9),
zone 4 comprised between the draw-off of the raffinate (9) and the injection of the desorbent (11),

The total number of beds of the separation unit (SMB) according to the invention is preferably comprised between 6 and 24 beds, and even more preferably between 8 and 15 beds distributed over one or more adsorbers.

The number of beds is adjusted so that each bed preferably has a height comprised between 0.70 m and 1.40 m.

The distribution of the quantity of solid adsorbent in each zone is as follows:
the quantity of solid adsorbent in zone 1 is 17%±5%,
the quantity of solid adsorbent in zone 2 is 42%±5%,
the quantity of solid adsorbent in zone 3 is 25%±5%,
the quantity of solid adsorbent in zone 4 is 17%±5%,

According to a preferred characteristic of the invention, it is possible to inject the desorbent and the feedstock into the separation unit (SMB), in a ratio by volume of desorbent to feedstock of at most 1.7/1 and preferably comprised between 1.5/1 and 0.4/1, inclusive.

The extract (6) is essentially constituted by toluene, paraxylene and desorbent.

The raffinate (9) is essentially constituted by toluene, metaxylene, orthoxylene, ethylbenzene, and paraxylene for the part not recovered in the extract, and desorbent.

The extract (6) is sent into a distillation column (EXT).

The desorbent (8) which is sent back into the separation unit (SMB) through the line (11) is drawn off at the bottom of the distillation column (EXT). At the top of the distillation column (EXT), a mixture of paraxylene and toluene is drawn off through the line (7).

The raffinate (9) is sent into a distillation column (RAF).

Desorbent (10) which is sent back into the separation unit (SMB) through the line (11) is drawn off at the bottom of the distillation column (RAF). A mixture of metaxylene, orthoxylene and ethylbenzene which is sent to the isomerization units (ISOM-1) and (ISOM-2) is drawn off through a line (12) at the top of the distillation column (RAF),

The flow (12) is divided into two flows (13) and (15), in proportions varying between 10-90 and 90-10 respectively, preferentially between 25-75 and 75-25, these proportions being percentages by weight.

The first isomerization zone (ISOM-1) operates preferably in liquid phase and is generally operated under the following conditions:
Temperature less than 300° C., preferably 200° C. to 260° C.,
Pressure less than 4 MPa, preferably 2 to 3 MPa,
Hourly space velocity (HSV) less than 10 h⁻¹ (10 litres per litre per hour), preferably comprised between 2 and 4 h⁻¹,
Catalyst comprising at least one zeolite having channels the opening of which is defined by a ring with 10 or 12 oxygen atoms (10 MR or 12 MR), preferentially a catalyst comprising at least one zeolite having channels the opening of which is defined by a ring with 10 oxygen atoms (10 MR), and even more preferably, a catalyst comprising a zeolite of ZSM-5 type.

The effluent from the isomerization unit (ISOM-1) is sent back through the line (14), either to the distillation column (S-1), or directly to the inlet of the separation unit (SMB) in the case where the content of compounds other than the C8 aromatics is very low, typically of the order of 1% by weight. The C9 content is typically less than 1000 ppm by weight.

The second isomerization unit (ISOM-2) operates in gas phase and is generally operated under the following conditions:
Temperature greater than 300° C., preferably 350° C. to 480° C.,
Pressure less than 4 MPa, preferably 0.5 to 2 MPa,
Hourly space velocity (HSV) less than 10 h⁻¹ (10 litres per litre per hour), preferably comprised between 0.5 and 6 h⁻¹,
Catalyst including at least one zeolite having channels the opening of which is defined by a ring with 10 or 12 oxygen atoms (10 MR or 12 MR), preferentially a catalyst comprising a zeolite of the EUO or MOR structure type, and at least one group VIII metal,
H₂/hydrocarbons molar ratio less than 10, and preferably comprised between 3 and 6.

All the catalysts capable of isomerizing the hydrocarbons with 8 carbon atoms, zeolitic or not, are suitable for the isomerization unit (ISOM-2) of the present invention. Preferably, a catalyst containing an acid zeolite, for example of the MFI, MOR, MAZ, FAU and/or EUO structure type is used. Even more preferably, a catalyst is used containing a zeolite of the EUO structure type and at least one metal from group VIII of the periodic table.

Preferably, the catalyst of the isomerization unit (ISOM-2) comprises from 1% to 70% by weight of a zeolite of the EUO structure type (EU-1 for example) comprising silicon and at least one element T preferably selected from aluminium and boron, the Si/T ratio of which is comprised between 5 and 100. Said zeolite is at least partially in the form of hydrogen, and the sodium content is such that the Na/T atomic ratio is less than 0.1. Optionally the catalyst of the isomerization unit can contain between 0.01 and 2% by weight of tin or indium, and sulphur at a content of 0.5 to 2 atoms per atom of the group VIII metal.

The effluent from the isomerization unit (ISOM-2) is sent into a separation system which makes it possible to recover a part of the hydrogen which is recycled to the isomerization unit (ISOM-2).

The non-recycled part of the hydrogen is made up by an addition of fresh hydrogen. At the end of the separation system an isomerate constituted by the heaviest fractions is recovered, which is sent back to the distillation column (S-1) through the line (16).

### EXAMPLES ACCORDING TO THE PRIOR ART AND ACCORDING TO THE INVENTION

### Example 1 (According to the Prior Art)

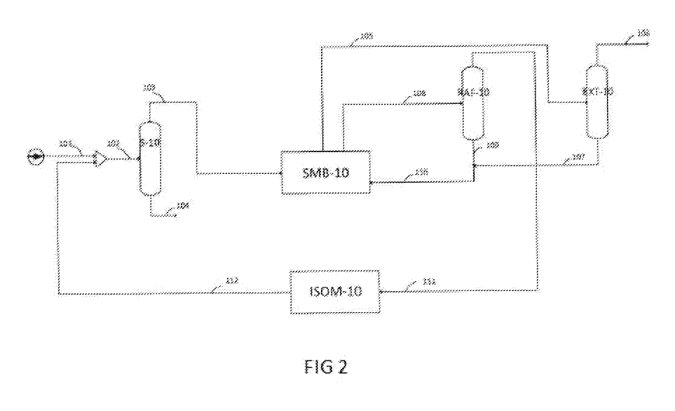

This example illustrates the prior art and describes an aromatic complex as shown in FIG. 2 and comprising:
a xylenes column (S-10) making it possible to extract the C9 and C10 aromatics (104) and to send a flow (103) essentially constituted by C8 aromatics to the separation unit (SMB-10),
a first simulated moving bed separation unit (SMB-10) with 4 zones from which an extract (105) and a single raffinate (108) are drawn off,
an isomerization unit (ISOM-10) supplied with a part (111) of the raffinate (108) after elimination of the desorbent (109) by means of the distillation column (RAF-10),
a paraxylene extraction column (EXT-10) from which the desorbent which is recycled to the adsorption unit (SMB-10) via the flow (110) is drawn off at the bottom and a cut rich in paraxylene (106) is drawn off at the top,

The material balance of the process is described in Table 1 below. Only the C8-aromatic and C9+ compounds are described. The other compounds and the formation of C9+ in the isomerization units are disregarded. The unit used for the flow rate is kilotonne per year (kt/yr).

TABLE 1

| | | PX | EB | MOX | C9+ | Total |
|---|---|---|---|---|---|---|
| Fresh feedstock | 101 | 23.6 | 15.6 | 67.7 | 13.8 | 120.6 |
| S-10 feedstock | 102 | 100 | 45.9 | 297 | 13.8 | 456.6 |
| SMB-10 feedstock | 103 | 100 | 45.9 | 297 | 0 | 442.9 |
| S-10 bottom | 104 | 0 | 0 | 0 | 13.8 | 13.8 |
| EXT-10 top | 106 | 100 | 0 | 0 | 0 | 100 |
| ISOM-10 feedstock | 11 | 0 | 45.9 | 297 | 0 | 342.9 |
| ISOM-10 outlet | 112 | 76.4 | 30.2 | 229.3 | 0 | 336.0 |

The feedstock (101) supplies the aromatic loop (mixture of the heavy reformate and toluene-column bottom) and has a flow rate of 120.4 kt/yr. 336 kt/yr of isomerate (112) recycled from the isomerization unit (ISOM-10) is added to the feedstock (101), isomerizing the ethylbenzene. The resulting flow (102) is distilled in the xylenes column (S-10).

13.8 kt/yr of a mixture of C9 and C10 aromatics (104) is drawn off at the bottom of the column (S-10) and 442.9 kt/yr of a cut of C8 aromatics (103) is drawn off at the top, of which the paraxylene content is 22.6%, the ethylbenzene content is 10.4%, and the orthoxylene and metaxylene content is 67%.

This cut is sent into a simulated moving bed separation unit with four zones (SMB-10) and four main flows: the feedstock (103), the desorbent (110), the extract (105) and the raffinate (108). This separation unit is composed of 12 beds containing an X zeolite exchanged with barium. The temperature is 175° C.

The configuration is:
2 beds in zone 1,
5 beds in zone 2,
3 beds in zone 3,
2 beds in zone 4.

The solvent used is paradiethylbenzene.

The extract (105) at the outlet of the adsorption unit (SMB-10) is sent into a distillation column (EXT-10) from which the desorbent recycled to the separation unit (SMB-10) is drawn off at the bottom, and 100 kt/yr of a mixture (106), essentially constituted by toluene and paraxylene, is drawn off at the top.

The raffinate is sent into a distillation column (RAF-10) from which the desorbent recycled to the adsorption unit (SMB-10) is drawn off at the bottom, and 342.9 kt/yr of a mixture (111) is drawn off at the top.

This flow is sent into an isomerization unit (ISOM-10).

The isomerization unit (ISOM-10) operates in gas phase under the following conditions:

Temperature: 385° C.

Catalyst: contains platinum and EU-1 zeolite

Hourly space velocity: 3.5 h⁻¹

H2/hydrocarbons ratio: 4.4:1

Pressure: 0.9 MPa

The ethylbenzene content of the mixture introduced into the isomerization unit (ISOM-10) is 13.4%.

A 2% loss by cracking is observed in this isomerization, i.e. a flow rate of 6.9 kt/yr. The ethylbenzene is partially isomerized, 9% of it remains in the outlet flow (112).

This isomerate (112) has a flow rate of 196 kt/yr. It is recycled to the inlet of the column (S-10) where it is mixed with the fresh feedstock (101) which has a flow rate of 120.9 kt/yr.

### Example 2 According to the Invention

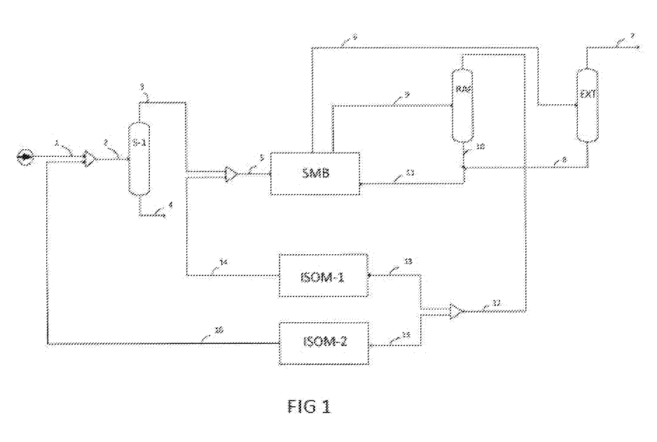

This example illustrates the invention and describes an aromatic loop shown in FIG. 1 and comprising:
a xylenes column (S-1) making it possible to extract the C9 and C10 aromatics (4) and to recover at the top a flow (3) essentially constituted by C8 aromatics,
a first simulated moving bed adsorption unit (SMB) with 4 zones from which an extract (6) and a raffinate (9) are drawn off,
a first paraxylene extraction column (EXT) from which the desorbent (8) which is recycled to the adsorption unit (SMB) via the flow (11) is drawn off at the bottom, and a cut rich in paraxylene (7) is drawn off at the top,
a first isomerization unit (ISOM-1) supplied with a first part of the raffinate (9) after elimination of the desorbent (10) by means of the distillation column (RAF),
a second isomerization unit (ISOM-2) supplied with a second part of the raffinate (9) after elimination of the desorbent (10) by means of the distillation column (RAF),

The material balance of the process is described in Table 2 below. Only the C8-aromatic and C9+ compounds are described. The other compounds and the formation of C9+ in the isomerization units are disregarded. The unit used for the flow rate is kilotonne per year (kt/yr).

TABLE 2

| | | PX | EB | MOX | C9+ | Total |
|---|---|---|---|---|---|---|
| Fresh feedstock | 1 | 22.8 | 15.3 | 65.5 | 13.4 | 117 |
| S-1 feedstock | 2 | 59.9 | 46.5 | 176.8 | 13.4 | 296.6 |
| S-1 top | 3 | 59.9 | 46.5 | 176.8 | 0 | 283.3 |
| S-1 bottom | 4 | 0 | 0 | 0 | 13.4 | 13.4 |
| SMB feedstock | 5 | 100 | 62.3 | 297 | 0 | 459.3 |
| EXT top | 7 | 100 | 0 | 0 | 0 | 100 |
| RAF top | 12 | 0 | 62.3 | 297 | 0 | 359.3 |
| ISOM-1 feedstock | 13 | 0 | 31.2 | 148.5 | 0 | 179.7 |
| ISOM-1 outlet | 14 | 37.1 | 31.2 | 111.4 | 0 | 179.7 |
| ISOM-2 feedstock | 15 | 0 | 31.2 | 148.5 | 0 | 179.7 |
| ISOM-2 outlet | 16 | 40.1 | 15.8 | 120.2 | 0 | 176.1 |

The fresh feedstock (1) which supplies the aromatic loop has a flow rate of 117 kt/yr.

176.1 kt/yr of isomerate (16) recycled from the isomerization unit (ISOM-2) is added to this feedstock, isomerizing the ethylbenzene. The resulting flow (2) is distilled in the xylenes column (S-1).

13.4 kt/yr of a mixture of C9 and C10 aromatics (4) is drawn off at the bottom of the distillation column (S-1) and 283.3 kt/yr of a cut of C8 aromatics (3) is drawn off at the top.

179.7 kt/yr of isomerate (14) recycled from the isomerization unit (ISOM-1) is added to this cut of C8 aromatics (3).

A mixture (5) is obtained, of which the paraxylene content is 21.8%, the ethylbenzene content is 13.6% and the orthoxylene and metaxylene content is 64.6%.

This cut is sent into a simulated moving bed adsorption unit with four zones (SMB) and four main flows: the feedstock (5), the desorbent (11), the extract (6) and the raffinate (9). This unit is composed of 12 beds containing an X zeolite exchanged with barium.

The temperature is 175° C. The configuration is: 2 beds in zone 1, 5 beds in zone 2, 3 beds in zone 3 and 2 beds in zone 4. The solvent used is paradiethylbenzene.

The extract (6) at the outlet of the adsorption unit (SMB) is sent into a distillation column (EXT) from which the desorbent (8) recycled to the adsorption unit (SMB) is drawn off at the bottom, and 100 kt/yr of a mixture (7) essentially constituted by toluene and paraxylene is drawn off at the top.

The raffinate (9) is sent into a distillation column (RAF) from which the desorbent (10) recycled to the adsorption unit (SMB) is drawn off at the bottom, and 359.3 kt/yr of a mixture (12) is drawn off at the top.

This flow is divided into two equal flows (13) and (15), each of 179.7 kt/yr.

The flow (13) is sent into an isomerization unit (ISOM-1).

The isomerization unit (ISOM-1) operates in liquid phase under the following conditions: Temperature: 240° C.

Catalyst: contains ZSM-5 zeolite

Hourly space velocity: 3 h⁻¹

Pressure: 1.9 MPa

The ethylbenzene content of the mixture introduced into the isomerization unit (ISOM-1) is 17.3%. The ethylbenzene is not converted; the quantity thereof is therefore the same in the outlet flow (14). This isomerate (14) has a flow rate of 179.7 kt/yr. It is recycled to the inlet of the adsorption unit (SMB) without passing through the column (S-1).

The flow (15) is sent into an isomerization unit (ISOM-2).

The isomerization unit (ISOM-2) operates in gas phase under the following conditions:

Temperature: 385° C.

Catalyst: contains platinum and EU-1 zeolite

Hourly space velocity: 3.5 h⁻¹

Pressure: 0.9 MPa

The ethylbenzene content of the mixture introduced into the isomerization unit (ISOM-2) is 17.3%. A 2% loss by cracking is observed in this isomerization, i.e. a flow rate of 3.6 kt/yr.

The ethylbenzene is partially isomerized. 9% of it remains in the outlet flow (16).

This isomerate (16) has a flow rate of 176.1 kt/yr. It is recycled to the inlet of the column (S-1) where it is mixed with the fresh feedstock (1) which has a flow rate of 117 kt/yr.

The invention has several advantages compared with the prior art:

Firstly, the liquid-phase isomerization unit consumes less energy than gas-phase isomerization. In fact, it operates at a lower temperature. It also operates without hydrogen recycling, therefore without a recycling compressor. Finally, it produces a much lower quantity of by-products, in particular of the C9 aromatics, which makes it possible to by-pass the C9 aromatics elimination column (S-1) greatly reducing the energy required for this separation. The fact of coupling a liquid-phase isomerization to a gas-phase isomerization makes it possible to reduce the losses by cracking within the gas-phase isomerization. In fact, in order to output 100 kt/yr of paraxylene, it is necessary to introduce 117 kt/yr of fresh feedstock in the invention as against 120.6 kt/an in the prior art.

1- Process for the production of high-purity paraxylene based on a xylenes cut containing ethylbenzene and C9+ compounds, a process using one simulated moving bed separation unit (SMB) and two isomerization units, one (ISOM-1) operating in liquid phase, and the other (ISOM-2) operating in gas phase, the process consisting of the sequence of the following stages:
the fresh feedstock (1) in a mixture with the isomerate (16) originating from the gas-phase isomerization unit (ISOM-2) is sent into the distillation column (S-1) from which a flow (3) which is mixed with the second isomerate (14) originating from the liquid-phase isomerization unit (ISOM-1) is removed at the top, and a flow (4) essentially constituted by C9 and C10 aromatic compounds and optionally orthoxylene is removed at the bottom.
a simulated mobile bed separation of the flow (5) resulting from the mixture of the flows (3) and (14) is carried out in a separation unit (SMB) comprising at least one adsorber containing a plurality of interconnected beds and operating in a closed loop, said separation unit comprising at least four zones defined as follows:
zone 1 comprised between the injection of the desorbent (11) and the draw-off of the extract (6),
zone 2 comprised between the draw-off of the extract (6) and the injection of the feedstock (5),
zone 3 comprised between the injection of the feedstock (5) and the draw-off of the intermediate raffinate (9),
zone 4 comprised between the draw-off of the raffinate (9) and the injection of the desorbent (11),
the extract 6 is sent into a distillation column (EXT), from which a mixture of paraxylene and toluene is drawn off at the top through the line (7) and the desorbent (8) which is sent back into the separation unit (SMB) is drawn off at the bottom through the line (11),
the raffinate 9 is sent into a distillation column (RAF), from which the desorbent (10) which is sent back into the separation unit (SMB) through the line (11) is drawn off at the bottom, and a mixture of metaxylene, orthoxylene and ethylbenzene which is sent through a line (12) to the isomerization units (ISOM-1 et ISOM-2) is drawn off at the top,
a first part of the flow (12), denoted flow (13), is sent into the liquid-phase isomerization unit (ISOM-1), in order to obtain a first isomerate (14), partially supplying the simulated moving bed separation unit (SMB), the isomerization unit (ISOM-1) operating in liquid phase under the following conditions:
temperature less than 300° C., preferably 200° C. to 260° C.,
pressure less than 4 MPa, preferably 2 to 3 MPa,
hourly space velocity (HSV) less than 10 h⁻¹ (10 litres per litre per hour), preferably comprised between 2 and 4 h⁻¹,
catalyst comprising at least one zeolite having channels the opening of which is defined by a ring with 10 or 12 oxygen atoms (10 MR or 12 MR), preferentially a catalyst comprising at least one zeolite having channels the opening of which is defined by a ring with 10 oxygen atoms (10 MR), and even more preferably, a catalyst comprising a zeolite of ZSM-5 type.
a second part of the flow (12), denoted flow (15), is sent into the gas-phase isomerization unit (ISOM-2), in order to obtain an isomerate (16), which is sent in a mixture with the fresh feedstock (1) into the distillation column (S-1), said gas-phase isomerization unit (ISOM-2) operating under the following conditions:
temperature greater than 300° C., preferably from 350° C. to 480° C.,
pressure less than 4.0 MPa and preferably from 0.5 to 2.0 MPa,
hourly space velocity less than 10 h⁻¹, preferably comprised between 0.5 h⁻¹ and 6 h⁻¹,
hydrogen to hydrocarbon molar ratio less than 10, and preferably comprised between 3 and 6,
and the catalyst used in said isomerization unit ISOM-2 comprising at least one zeolite having channels the opening of which is defined by a ring with 10 to 12 oxygen atoms (10 MR or 12 MR), and at least one group VIII metal at a content comprised between 0.1 and 0.3% by weight, inclusive.

2- Process for the production of high-purity paraxylene according to claim 1, in which the catalyst used in the isomerization unit (ISOM-2) contains from 1 to 70% by weight of a zeolite of the EUO structure type (EU-1 for example) comprising silicon and at least one element T preferably selected from aluminium and boron, the Si/T ratio of which is comprised between 5 and 100.

3- Process for the production of high-purity paraxylene according to claim 1, in which the zeolite forming part of the isomerization unit (ISOM-2) is at least partially in the form of hydrogen, and the sodium content is such that the Na/T atomic ratio is less than 0.1.

4- Process for the production of high-purity paraxylene according to claim 1, in which the catalyst of the isomerization unit (ISOM-2) can contain between 0.01 and 2% by weight of tin or indium, and sulphur at a content of 0.5 to 2 atoms per atom of the group VIII metal.

5- Process for the production of high-purity paraxylene according to claim 1, in which the total number of beds of the separation unit (SMB) is comprised between 6 and 24 beds, and preferably between 8 and 15 beds, distributed over one or more adsorbers, the number of beds being adjusted so that each bed has a height comprised between 0.70 m and 1.40 m.

6- Process for the production of high-purity paraxylene according to claim 1, in which the distribution of the quantity of solid adsorbent in each zone of the separation unit (SMB) is as follows:
the quantity of solid adsorbent in zone 1 is 17%±5%,
the quantity of solid adsorbent in zone 2 is 42%±5%,
the quantity of solid adsorbent in zone 3 is 25%±5%,
the quantity of solid adsorbent in zone 4 is 17%±5%,

7- Process for the production of high-purity paraxylene according to claim 1, in which the desorbent and the feedstock are injected into the separation unit (SMB) in a desorbent to feedstock ratio by volume of at most 1.7/1 and preferably comprised between 1.5/1 and 0.4/1, inclusive.

8- Process for the production of high-purity paraxylene according to claim 1, in which two raffinates (R1) and (R2) are extracted from the separation unit (SMB), (R1) being sent to the isomerization unit (ISOM-1) and (R2) being sent into the isomerization unit (ISOM-2).
